(19) United States
(12) Patent Application Publication
(10) Pub. No.: US 2014/0221480 A1
(43) Pub. Date: Aug. 7, 2014

(54) METHODS OF MANAGING BRAIN INFLAMMATION

(71) Applicants: Fang Hua, Lilburn, GA (US); Donald G. Stein, Atlanta, GA (US); Iqbal Sayeed, Atlanta, GA (US)

(72) Inventors: Fang Hua, Lilburn, GA (US); Donald G. Stein, Atlanta, GA (US); Iqbal Sayeed, Atlanta, GA (US)

(73) Assignee: EMORY UNIVERSITY, Atlanta, GA (US)

(21) Appl. No.: 14/173,942

(22) Filed: Feb. 6, 2014

Related U.S. Application Data
(60) Provisional application No. 61/761,272, filed on Feb. 6, 2013.

(51) Int. Cl. A61K 31/24

(52) U.S. Cl. CPC A61K 31/24
USPC 514/538

(57) ABSTRACT

This disclosure relates to methods of managing brain injury such as inflammation due to trauma induced brain injury and ischemic stroke by administering resatorvid or derivative there of a subject in need thereof. In certain embodiments, the disclosure relates to methods of treating cerebral ischemia comprising administering an effective amount of resatorvid to a subject in need thereof. In certain embodiments, the effective amount is 3 mg/kg. In certain embodiments, resatorvid is administered by injection.

### CROSS-REFERENCE TO RELATED APPLICATIONS

This Application claims priority to U.S. Provisional Application No. 61/761,272 filed Feb. 6, 2013, hereby incorporated by reference in its entirety.

### STATEMENT REGARDING FEDERALLY FUNDED RESEARCH

This invention was made with government support under Grant 5R01NS048451 and 1R01HD061971 awarded by the National Institutes of Health. The government has certain rights in the invention.

### BACKGROUND

Brain injuries, including traumatic brain injury (TBI) and stroke, affect well over 2 million Americans each year and are a significant health concern worldwide. Traumatic brain injuries result from a blow or jolt to the head or a penetrating head injury that disrupts the function of the brain, with severity ranging from “mild,” i.e., a brief change in mental status or consciousness to “severe,” i.e., an extended period of unconsciousness or amnesia after the injury. In contrast, strokes are a result of diseases that affect the blood vessels that supply blood to the brain. A stroke occurs when a blood vessel that brings oxygen and nutrients to the brain either bursts (hemorrhagic stroke) or is clogged by a blood clot or some other mass (ischemic stroke). The majority of strokes are ischemic, however hemorrhagic strokes typically result in more severe injuries.

Despite several decades of effort, scientists have not yet found a pharmacological agent that consistently improves outcomes after stroke or TBI (see Sauerland, S. et al., Lancet 2004, 364, 1291-1292; Brain Trauma Foundation, American Association of Neurological Surgeons, Joint Section on Neurotrauma and Critical Care. Guidelines for the management of severe head injury, J. Neurotrauma 1996, 13, 641-734).

After TBI or stroke, inflammation is a principle cause of secondary damage and long-term damage. Following insults to the central nervous system, a cascade of physiological events leads to neuronal loss including, for example, an inflammatory immune response and excitotoxicity resulting from disrupting the glutamate, acetylcholine, cholinergic, GABAA, and NMDA receptor systems. In these cases, a complex cascade of events leads to the delivery of blood-borne leucocytes to sites of injury to kill potential pathogens and promote tissue repair. However, the powerful inflammatory response has the capacity to cause damage to normal tissue, and dysregulation of the innate, or acquired immune response is involved in different pathologies.

In addition to TBI and stroke, inflammation is being recognized as a key component of a variety of nervous system disorders. It has long been known that certain diseases such as multiple sclerosis result from inflammation in the central nervous system, but it is only in recent years that it has been suggested that inflammation may significantly contribute to neurodegenerative disorders such as HIV-related dementia, Alzheimer's and prion diseases. It is now known that the resident macrophages of the central nervous system (CNS), the microglia, when activated may secrete molecules that cause neuronal dysfunction, or degeneration.

TAK-242, a cyclohexene derivative, is a small-molecule that selectively inhibits TLR4 signaling. See Ii et al. TAK-242 selectively inhibits toll-like receptor 4-mediated cytokine production through suppression of intracellular signaling. Mol Pharmacol, 2006, 69(4):1288-95. Matsunaga et al., report TAK-242 (resatorvid) binds selectively to TLR4 and interferes with interactions between TLR4 and its adaptor molecules. Mol Pharmacol, 2011, 79(1):34-41. Sha et al. report combination of imipenem and TAK-242 improves survival in a murine model of polymicrobial sepsis. Shock, 2011, 35(2):205-209.

Kawamoto et al. report TAK-242 selectively suppresses Toll-like receptor 4-signaling mediated by the intracellular domain. Eur J Pharmacol, 2008, 584(1):40-8.

Hua et al. report differential roles of TLR2 and TLR4 in acute focal cerebral ischemia/reperfusion injury in mice. Brain Res, 2009, 1262:100-8.

Caso et al. report toll-like receptor 4 is involved in brain damage and inflammation after experimental stroke. Circulation, 2007, 115: 1599-1608. See also Stroke, 2008, 39: 1314-1320 entitled “Toll-Like Receptor 4 Is Involved in Subacute Stress-Induced Neuroinflammation and in the Worsening of Experimental Stroke.”

U.S. Published Application Number 2009/0215908 reports toll like receptor signaling antagonists. See also U.S.2010/0239523 and U.S.2003/0077279.

Suzuki et al. report pharmacological inhibition of TLR4-NOX4 signal protects against neuronal death in transient focal ischemia. Scientific Reports, 2012, 2:896.

Nilsson e al. report Soluble TNF receptors are associated with infarct size and ventricular dysfunction in ST-elevation myocardial infarction. PLoS One, 2013, 8(2):e55477

References cited herein are not an admission of prior art.

### SUMMARY

This disclosure relates to methods of treating or preventing brain injury such as inflammation due to trauma induced brain injury and ischemic stroke by administering resatorvid or derivative there of a subject in need thereof. In certain embodiments, the disclosure relates to methods of treating cerebral ischemia comprising administering an effective amount of resatorvid to a subject in need thereof. In certain embodiments, the effective amount is 3 mg/kg. In certain embodiments, resatorvid is administered by injection.

In certain embodiments, the disclosure relates to methods of treating traumatic brain injury comprising administering an effective amount of ethyl (6R)-6-[N-(2-chloro-4-fluorophenyl)sulfamoyl]cyclohex-1-ene-1-carboxylate or derivative thereof to a subject at risk of, exhibiting symptoms of, or diagnosed with ischemic stroke. In certain embodiments, the subject is a human subject. In certain embodiments, the effective amount is between 1 to 5 mg/kg body weight. In certain embodiments, the effective amount is between 2 to 4 mg/kg body weight. In certain embodiments, the effective amount is between 100 mg to 500 mg. In certain embodiments, the effective amount is between 200 mg to 400 mg. In certain embodiments, the administration is oral, intraperitoneal, or intravenous injection.

In certain embodiments, the disclosure relates to methods of treating ischemic stroke comprising administering an effective amount of ethyl (6R)-6-[N-(2-chloro-4-fluorophenyl)sulfamoyl]cyclohex-1-ene-1-carboxylate or derivative thereof to a subject at risk of, exhibiting symptoms of, or diagnosed with ischemic stroke. In certain embodiments, the subject is a human subject. In certain embodiments, the effective amount is between 1 to 5 mg/kg body weight. In certain embodiments, the effective amount is between 2 to 4 mg/kg body weight. In certain embodiments, the effective amount is between 100 mg to 500 mg. In certain embodiments, the effective amount is between 200 mg to 400 mg. In certain embodiments, the administration is oral, intraperitoneal, or intravenous injection.

In certain embodiments, the derivative is (6R)-6-[N-(2-chloro-4-fluorophenyl)sulfamoyl]cyclohex-1-ene-1-carboxylic acid or alkyl ester thereof optionally substituted with one or more substituents. These compounds may be used for any uses or methods reported herein.

In certain embodiments, the disclosure relates to pharmaceutical compositions comprising ethyl (6R)-6-[N-(2-chloro-4-fluorophenyl)sulfamoyl]cyclohex-1-ene-1-carboxylate or derivative thereof and a pharmaceutically acceptable excipient. The pharmaceutical composition may optionally further comprise a second active therapeutic agent.

In certain embodiments, the disclosure relates to methods of treating or preventing inflammation comprising administering an effective amount of a compound or a pharmaceutical composition comprising a compound disclosed herein to a subject in need thereof. In certain embodiments, the pharmaceutical composition is administered to a subject that incurred trauma to the head or other organ or tissue. In certain embodiments, the pharmaceutical composition is administered after a medical procedure. In certain embodiments, the pharmaceutical composition is administered in combination with a second anti-inflammatory agent.

In certain embodiments, the disclosure relates to methods of treating stroke or traumatic brain injury comprising administering an effective amount of a compound or a pharmaceutical composition comprising a compound disclosed herein to a subject in need thereof.

In other embodiments, the disclosure relates to methods of treating or preventing neurodegeneration resulting from ischemic CNS injuries, in particular from ischemic stroke comprising administering a compound(s) or pharmaceutical composition(s) disclosed herein to a patient in need thereof.

In certain embodiments, the disclosure relates to methods of treating or preventing a neurodegenerative disease or condition comprising administering an effective amount of a pharmaceutical composition to a subject in need thereof, e.g., at risk of, exhibiting symptoms of, or diagnosed with the disease or condition

Pharmaceutical compositions, including in combination with additional neuroprotective agents, are also provided.

In certain embodiments, the disclosure relates to the production of a medicament for uses disclosed herein.

In certain embodiment, the disclosure relates to compounds disclosed herein and derivatives such as the compounds substituted with one or more substituents and salts thereof.

### BRIEF DESCRIPTION OF THE FIGURES

FIG. 1 shows data on the concentration of TAK-242 in plasma and brain tissue after injection (3 mg/kg body weight). The concentration of TAK-242 in plasma (red line) increased to 52.0 ng/ml 3 hrs after injection, was maintained at 54.1 ng/ml 8 hrs after injection, and decreased to 22.6 ng/ml 24 hrs after injection. The concentration of TAK-242 in the hemisphere contralateral to ischemia (green line) increased to 14.2 ng/ml 3 hrs after injection, was maintained at 15.1 ng/ml 8 hrs after injection, and was still maintained at 17.5 ng/ml (contralateral) 24 hrs after injection. The concentration of TAK-242 in ischemic hemisphere (blue line) increased to 26.1 ng/ml 3 hrs after injection, was maintained at 26.4 ng/ml 8 hrs after injection, and was still maintained at 25.0 ng/ml 24 hrs after injection. The concentrations of TAK-242 in ischemic hemisphere (blue line) were significantly higher than those in contralateral hemisphere (green line) (*p<0.05).

FIG. 2 shows data on brain infarct size and Neurological Score 24 hrs after cerebral ischemia. The infarct size was 21.3% in control group, and 12.5% in TAK-242 treated group. TAK-242 treatment significantly reduced brain infarct size by 41% compared to control mice (#p<0.05).

The neurological score was 4.38 in the control group, and 6.73 in the TAK-242-treated group. TAK-242 treatment significantly improved neurological function by 34% compared to control mice (#p<0.05). A representative picture of TTC staining is shown on the top of FIG. 2.

FIG. 3 shows data on levels of sTNF RI, KC, GSCF, and IL-6 in brain tissue 6 hrs after cerebral Ischemia. The levels of sTNFRI, KC, GSCF, and IL-6 significantly increased in ischemic brain compared with sham controls (#p<0.05). (Sham: sham control; FR: cerebral ischemia/reperfusion; S-Tak: sham control with TAK-242; I/R-Tak: I/R treated with TAK-242).

FIG. 4 shows data on levels of sTNF RII, MCP-1, MIP-1γ, and TIMP-1 in brain tissue 6 hrs after cerebral ischemia. Levels of sTNFRII, MCP-1, MIP-1γ, and TIMP-1 significantly increased in ischemic brain compared with sham controls (#p<0.05). Treatment with TAK-242 significantly reduced the levels of these cytokines compared with untreated controls (*p<0.05). (Sham: sham control; I/R: cerebral ischemia/reperfusion; S-Tak: sham control with TAK-242; I/R-Tak: I/R treated with TAK-242).

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

Prior to describing the various embodiments, the following definitions are provided and should be used unless otherwise indicated.

As used herein, the term “compound” refers to ethyl (6R)-6-[N-(2-chloro-4-fluorophenyl)sulfamoyl]cyclohex-1-ene-1-carboxylate or derivative thereof.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

As used herein, “alkyl” means a noncyclic straight chain or branched, unsaturated or saturated hydrocarbon such as those containing from 1 to 10 carbon atoms, while the term “lower alkyl” or “C1-4alkyl” has the same meaning as alkyl but contains from 1 to 4 carbon atoms. The term “higher alkyl” has the same meaning as alkyl but contains from 7 to 20 carbon atoms. Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

Non-aromatic mono or polycyclic alkyls are referred to herein as “carbocycles” or “carbocyclyl” groups. Representative saturated carbocycles include cyclopropyl, cyclobutyl, cyclopentyl, cyclohexyl, and the like; while unsaturated carbocycles include cyclopentenyl and cyclohexenyl, and the like.

“Heterocarbocycles” or heterocarbocyclyl” groups are carbocycles which contain from 1 to 4 heteroatoms independently selected from nitrogen, oxygen and sulfur which may be saturated or unsaturated (but not aromatic), monocyclic or polycyclic, and wherein the nitrogen and sulfur heteroatoms may be optionally oxidized, and the nitrogen heteroatom may be optionally quaternized. Heterocarbocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Aryl” means an aromatic carbocyclic monocyclic or polycyclic ring such as phenyl or naphthyl. Polycyclic ring systems may, but are not required to, contain one or more non-aromatic rings, as long as one of the rings is aromatic.

As used herein, “heteroaryl” refers an aromatic heterocarbocycle having 1 to 4 heteroatoms selected from nitrogen, oxygen and sulfur, and containing at least 1 carbon atom, including both mono- and polycyclic ring systems. Polycyclic ring systems may, but are not required to, contain one or more non-aromatic rings, as long as one of the rings is aromatic. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes N-alkylated derivatives such as a 1-methylimidazol-5-yl substituent.

As used herein, “heterocycle” or “heterocyclyl” refers to mono- and polycyclic ring systems having 1 to 4 heteroatoms selected from nitrogen, oxygen and sulfur, and containing at least 1 carbon atom. The mono- and polycyclic ring systems may be aromatic, non-aromatic or mixtures of aromatic and non-aromatic rings. Heterocycle includes heterocarbocycles, heteroaryls, and the like.

“Alkylthio” refers to an alkyl group as defined above attached through a sulfur bridge. An example of an alkylthio is methylthio, (i.e., —S—CH3).

“Alkoxy” refers to an alkyl group as defined above attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, i-propoxy, n-butoxy, s-butoxy, t-butoxy.

“Alkylamino” refers an alkyl group as defined above attached through an amino bridge. An example of an alkylamino is methylamino, (i.e., —NH—CH3). “Alkanoyl” refers to an alkyl as defined above attached through a carbonyl bride (i.e., —(C═O)alkyl).

“Alkylsulfonyl” refers to an alkyl as defined above attached through a sulfonyl bridge (i.e., —S(═O)2alkyl) such as mesyl and the like, and “Arylsulfonyl” refers to an aryl attached through a sulfonyl bridge (i.e., —S(=O)2aryl).

“Alkylsulfinyl” refers to an alkyl as defined above attached through a sulfinyl bridge (i.e. —S(═O)alkyl).

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(=O)NRaNRb, —NRaC(═O)ORb, —NRaSO2Rb, —C(═O)Ra, —C(═O)ORa, —C(═O)NRaRb, —OC(═O)NRaRb, —ORa, —SRa, —SORa, —S(═O)2Ra, —OS(═O)2Ra and —S(═O)2ORa. Ra and Rb in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

The term “optionally substituted,” as used herein, means that substitution is optional and therefore it is possible for the designated atom to be unsubstituted.

As used herein, “salts” refer to derivatives of the disclosed compounds where the parent compound is modified making acid or base salts thereof. Examples of salts include, but are not limited to, mineral or organic acid salts of basic residues such as amines, alkylamines, or dialkylamines; alkali or organic salts of acidic residues such as carboxylic acids and phosphates; and the like. In preferred embodiment the salts are conventional nontoxic pharmaceutically acceptable salts including the quaternary ammonium salts of the parent compound formed, and non-toxic inorganic or organic acids. Preferred salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. In certain embodiments, the compounds herein are phosphate salts with a positive one or two metal cation such as sodium, lithium, potassium, quaternary ammonium, calcium, magnesium, or combinations thereof.

“Subject” refers any animal, preferably a human patient, livestock, rodent, primate, monkey, or domestic pet.

The term “prodrug” refers to an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis.

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, replacing an oxygen atom with a sulfur or nitrogen atom or replacing an amino group with a hydroxyl group or vice versa. The derivative may be a prodrug. Derivatives may be prepare by any variety of synthetic methods or appropriate adaptations presented in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze.

### TAK-242, an Antagonist for Toll-Like Receptor 4, Protects Against Acute Cerebral Ischemia/Reperfusion Injury

Whether TAK-242 can pass through blood brain barrier (BBB) and inhibit neuroinflammation in ischemic brain has not been investigated. Studies were performed to evaluate the ability of TAK-242 to pass through the BBB, the protective effect of TAK-242 on ischemic brain, and the modulation of TLR4 on inflammatory cytokines in mice subjected to acute cerebral I/R.

TLR4 is known to be involved in cerebral ischemia/reperfusion (I/R) injury and is considered a potential target for the treatment of ischemic stroke. A mouse model of cerebral FR was induced by transient middle cerebral artery occlusion (tMCAO). TAK-242 (3 mg/kg body weight) was injected intraperitoneally (i.p.) 1 hour after ischemia. Concentrations of TAK-242 in plasma and brain tissue were measured 3, 8, and 24 hrs after injection. Neurological scores were evaluated 24 hrs after cerebral FR. Brain infarct areas were detected by 2,3,5-triphenyltetrazolium chloride (TTC) staining Inflammatory cytokines were analyzed by antibody arrays 6 hrs after cerebral I/R. The concentration of TAK-242 in plasma increased 3 hrs after treatment, was maintained at a high level 8 hrs after treatment, and decreased 24 hrs after treatment. The concentration of TAK-242 in brain increased 3 hrs after treatment and was still maintained at a high level at 24 hrs. TAK-242 treatment significantly reduced brain infarct size (12.5%) compared to vehicle control mice (21.3%) (p<0.05). Following ischemic injury the levels of sTNF RI, sTNF RII, KC, GSCF, IL-6, MCP-1, MIP-1γ, and TIMP-1 were all increased. TAK-242 treatment significantly reduced the levels of sTNF RII, MCP-1, MIP-1γ, and TIMP-1 (p<0.05). TAK-242 passes the blood-brain barrier and treatment with the drug protects the brain from acute damage after cerebral I/R by mediating the expression of inflammatory cytokines.

### Methods of Use

In certain embodiments, the disclosure relates to methods and compositions for the treatment or prevention of neurodegeneration following an injury to the central nervous system or due to certain neurodegenerative disorders comprising administering an effective amount of a compound described herein, or a pharmaceutically acceptable salt, ester or prodrug thereof to a subject in need thereof. Multiple physiological events lead to neurodegeneration. These events include, for example, increase in the immune and inflammatory response, demyelinization, and lipid peroxidation. In certain embodiments, the disclosure relates to compositions and methods for reducing or eliminating neuronal cell death, edema, ischemia, and enhancing tissue viability following injury to the central nervous system or certain disorders. The analogues, salts, esters or prodrugs of the, compound, steroid, or secosteroid analogs may be optionally administered with a pharmaceutically acceptable carrier or diluent.

As used herein, “neuroprotection” is the prevention, arrest or reverse progression of neurodegeneration following a central nervous system injury. The neuroprotective effect includes both improved morphological (i.e., enhanced tissue viability) and/or behavioral recovery. CNS injuries that are encompassed within the scope of treatment include both traumatic injuries, in particular TBI, and physiological insults such as an ischemic or hemorrhagic stroke. In both instances, a progressive loss of neurons after the initial insult occurs and can be alleviated.

In certain embodiments, the disclosure relates to methods of preventing or reducing inflammatory reactions in a patient by administering a compound disclosed herein to a subject in need thereof. In certain embodiments, methods of neuroprotection are provided comprising administering a compound disclosed herein, its physiologically acceptable salt or prodrug, optionally in a pharmaceutically acceptable carrier, to a patient at risk of suffering from a stroke. In other embodiments, methods of treating or preventing neuronal damage are provided comprising administering a compound disclosed herein or its physiologically acceptable salt or prodrug, optionally in a pharmaceutically acceptable carrier, to a patient who has suffered from an ischemic stroke. The method can reduce or prevent neurodegeneration such as that caused by excitotoxic or inflammatory reactions, or can enhance neuronal proliferation, growth or differentiation in the period after the injury. In yet further embodiments, methods of treating or preventing cognitive or behavioral deficits after a stroke is provided comprising administering a compound disclosed herein or its physiologically acceptable salt or prodrug, optionally in a pharmaceutically acceptable carrier, to a human subject who has suffered a stroke. In certain embodiments, the stroke is an ischemic stroke, but it can alternatively be a hemorrhagic stroke.

In yet other embodiments, the disclosure relates to methods of treating or preventing neurodegeneration resulting from hemorrhagic CNS injuries, in particular from hemorrhagic stroke comprising administering a compound disclosed herein to a patient in need thereof. The methods can alleviate the initial damage to the CNS. Therefore, in some embodiments, the compounds are administered to a patient at risk of a CNS injury, in particular to a patient at risk of a stroke. The compounds are also effective at reducing or preventing secondary injuries. Therefore, in other embodiments, the compounds are administered to a patient who has suffered a CNS injury within a window of opportunity after the initial insult. The initial insult can be either a TBI or a stroke, whether that be an ischemic or hemorrhagic stroke.

In other embodiments, the present disclosure relates to methods to achieve a neuroprotective effect following a traumatic CNS injury in a mammal, in particular in a human, comprising administering a therapeutically effective amount of a compound disclosed herein. A traumatic injury to the CNS is characterized by a physical impact to the central nervous system. The physical forces resulting in a traumatic brain injury cause their effects by inducing three types of injury: skull fracture, parenchymal injury, and vascular injury. A blow to the surface of the brain typically leads to rapid tissue displacement, disruption of vascular channels, and subsequent hemorrhage, tissue injury and edema. Morphological evidence of injury in the neuronal cell body includes pyknosis of nucleus, eosinophilia of the cytoplasm, and disintegration of the cell. Furthermore, axonal swelling can develop in the vicinity of damage neurons and also at great distances away from the site of impact.

In certain embodiments, the compound is administered within twelve hours after onset of a stroke. In certain embodiments, the compound is administered within twelve hours after an injury, such as a TBI. In some embodiments, the compound is administered within 11 hours of a TBI, stroke or other injury to the brain, or within 10 hours, or within 9 hours, or within 8 hours, or within 7 hours, or within 6 hours, or within 5 hours, or within 4 hours, or within 3 hours, such as within two or one hour. In some embodiments, the compounds are administered within one day (i.e. 24 hours) of the injury. In certain embodiments, the compounds are provided to individuals at risk of a stroke, such as those who are suffering from atherosclerosis or have a family history of heart disease. These compounds can be provided to individuals as a preventative therapy to decrease neural trauma.

In another embodiment, a method for decreasing ischemia following a brain injury is provided comprising administering an effective amount of a compound disclosed herein. Although it is not intended that embodiments of the disclosure work by any particular mechanism, it is believed that administering certain compound is a means to reduce or eliminate the inflammatory immune reactions that follow a CNS injury. By reducing the inflammatory response, the compounds can substantially reduce brain swelling and reduce the amount of neurotoxic substances (e.g., free radicals and excitotoxins) that are released from the site of injury.

In certain embodiments, the concentration of the compound or salt, ester or prodrug thereof, is effective in the treatment or prevention of typical neuronal damage that follows either a traumatic, ischemic or hemorrhagic injury to the CNS and hence, elicits a neuroprotective effect. The therapeutically effective amount will depend on many factors including, for example, the specific activity of the compound administered, the type of injury, the severity and pattern of the injury, the resulting neuronal damage, the responsiveness of the patient, the weight of the patient along with other intraperson variability, the method of administration, and the formulation used.

It is recognized that a traumatic injury to the CNS results in multiple physiological events that impact the extent and rate of neurodegeneration, and thus the final clinical outcome of the injury. The treatment of a traumatic injury to the CNS encompasses any reduction and/or prevention in one or more of the various physiological events that follow the initial impact. For example, cerebral edema frequently develops following a traumatic injury to the CNS and is a leading cause of death and disability. Cortical contusions, for example, produce massive increases in brain tissue water content which, in turn, can cause increased intracranial pressure leading to reduced cerebral blood flow and additional neuronal loss. Hence, the methods disclosed herein find use in reducing and/or eliminating cerebral edema and/or reducing the duration of the edemic event following a traumatic injury to the CNS.

Further physiological effects of brain injury include an inflammatory response. In particular, some studies indicate that the acute inflammatory response contributes significantly to injury after ischemia (see Perera, et al. (2005) Inflammation following stroke. J. CHn. Neurosc. 13:1-8; Barone and Feuerstein (1999) Inflammatory mediators and stroke: new opportunities for novel therapeutics). The stroke process triggers an inflammatory reaction that may last up to several months. Suppression of inflammation can reduce infarct volume and improve clinical outcomes even with the initiation of therapy after 3 hours of onset of stroke. In addition, an immune response can be triggered both by strokes. Infiltrating leukocytes are thought to contribute to secondary ischemic damage by producing toxic substances that kill brain cells and disrupt the blood-brain barrier (see del Zoppo, et al. (2000) Advances in the vascular pathophysiology of ischemic stroke. Thromb Res. 98:73-81) Infiltration occurs when leukocytes bind endothelial intercellular adhesion molecule-1 (ICAM-I) and ICAM-I is upregulated after ischemia.

TBI also elicits inflammatory, and in particular an immune responses. See, for example, Soares et al. (1995) J. Neurosci. 15:8223-33; Holmin et al. (1995) Acta Neurochir. 132:110-9; Arvin et al. (1996) Neurosci. Biobehav. Rev. 20:445-52. Following a cortical impact, severe inflammatory reactions and gliosis at the impact site and at brain areas distal to the primary site of injury occurs. The inflammatory response is characterized by the expression of adhesion molecules on the vascular surfaces, resulting in the adherence of immune cells and subsequent extravasation into the brain parenchyma. By releasing cytokines, the invading macrophages and neutrophils stimulate reactive astrocytosis. Release of different chemokines by other cell types induces these immune cells to become phagocytic, with the simultaneous release of free radicals and pro-inflammatory compounds, e.g., cytokines, prostaglandins, and excitotoxins (Arvin et al. (1996) Neurosci. Biobehav. Ref 20:445-52; Raivich et al. (1996) Kelo J. Med. 45:239-47; Mattson et al. (1997) Brain Res. Rev. 23:47-61).

Assays for assessing the efficacy of the compounds described herein include assays to determine a decrease in an ischemic event include, for example, a decrease in infarct area, improved body weight, and improved neurological outcome. Assays to measure a reduction in lipid peroxidation in both brain homogenate and in mitochondria are known in the art and include, for example, the thiobarbituric acid method (Roof et al. (1997) MoI. Chem. Neuropathol. 31:1-11; Subramanian et al. (1993) Neurosci. Lett. 155:151-4; Goodman et al. (1996) J. Neurochem. 66:1836-44; Vedder et al. (1999) J. Neurochem. 72:2531-8) and various in vitro free radical generating systems. Furthermore, alterations in the levels of critical free radical scavenger enzymes, such as mitochondrial glutathione can be assayed. See, for example, Subramanian et al. (1993) Neurosci. Lett. 155:151-4; and Vedder et al. (1999) J. Neurochem. 72:2531-8.

In addition, behavioral assays can be used to determine the rate and extent of behavior recovery in response to the treatment. Improved patient motor skills, spatial learning performance, cognitive function, sensory perception, speech and/or a decrease in the propensity to seizure may also be used to measure the neuroprotective effect. Such functional/behavioral tests used to assess sensorimortor and reflex function are described in, for example, Bederson et al. (1986) Stroke 17:472-476, DeRyck et al. (1992) Brain Res. 573:44-60, Markgraf et al. (1992) Brain Res. 575:238-246, Alexis et al. (1995) Stroke 26:2336-2346 Enhancement of neuronal survival may also be measured using the Scandinavian Stroke Scale (SSS) or the Barthl Index. Behavioral recovery can be further assessed using the recommendations of the Subcommittee of the NIH/NINDS Head Injury Centers in Humans (Hannay et al. (1996) J. Head Trauma Rehabil. 11:41-50). Behavioral recovery can be further assessed using the methods described in, for example, Beaumont et al. (1999) Neural Res. 21:742-754; Becker et al. (1980) Brain Res. 200:07-320; Buresov et al. (1983) Techniques and Basic Experiments for the Study of Brain and Behavior; Kline et al. (1994) Pharmacol. Biochem. Behav. 48:773-779; Lindner et al. (1998) J. Neurotrauma 15:199-216; Morris (1984) J. Neurosci. Methods 11:47-60; Schallert et al. (1983) Pharmacol. Biochem. Behav. 18:753-759.

Furthermore, a reduction in the inflammatory immune reactions following a traumatic brain injury can be assayed by measuring the cytokines level following the injury in the sham controls versus the treated subjects. Cytokines are mediators of inflammation and are released in high concentrations after brain injury. The level of pro-inflammatory cytokines (e.g., interleukin 1-beta, tumor necrosis factor, and interleukin 6) and the level of anti-inflammatory cytokines (e.g., interleukin 10 and transforming growth factor-beta) can be measured. For instance, “real-time” polymerase chain reactions (PCR) can be used to measure the strength of the mRNA signal and ELISA can be used to determine protein levels. In addition, histological analysis for different inflammatory cell types (e.g., reactive astrocytes, macrophages and microglia) can be used to measure a reduction in the inflammatory response.

In certain embodiments, the disclosure contemplates methods of treating or preventing brain injury such as inflammation due to trauma induced brain injury and ischemic stroke by administering resatorvid or derivative thereof in combination with other therapeutic agents, e.g., neuroprotective agents, anti-inflammatory agents, and/or anti-thrombotic agents to a subject in need thereof.

In certain embodiments, resatorvid or derivative thereof is administered in combination with progesterone. In certain embodiments, resatorvid or derivative thereof is administered in combination with allopregnanolone. In certain embodiments, resatorvid or derivative thereof is administered in combination with testosterone. In certain embodiments, resatorvid or derivative thereof is administered in combination with estrogen. In certain embodiments, resatorvid or derivative thereof is administered in combination with fludrocortisone.

In certain embodiments, resatorvid or derivative thereof is administered in combination with recombinant tissue plasminogen activator (rtPA). In certain embodiments, resatorvid or derivative thereof is administered in combination with recombinant human growth hormone. In certain embodiments, resatorvid or derivative thereof is administered in combination with citicoline. In certain embodiments, resatorvid or derivative thereof is administered in combination with sertraline. In certain embodiments, resatorvid or derivative thereof is administered in combination with duloxetine. In certain embodiments, resatorvid or derivative thereof is administered in combination with propranolol. In certain embodiments, resatorvid or derivative thereof is administered in combination with armodafinil. In certain embodiments, resatorvid or derivative thereof is administered in combination with enoxaparin. In certain embodiments, resatorvid or derivative thereof is administered in combination with autologous bone marrow mononuclear cell transplantation. In certain embodiments, resatorvid or derivative thereof is administered in combination with hypertonic saline and/or mannitol. In certain embodiments, resatorvid or derivative thereof is administered in combination with buspirone. In certain embodiments, resatorvid or derivative thereof is administered in combination with atorvastatin. In certain embodiments, resatorvid or derivative thereof is administered in combination with rivastigmine In certain embodiments, resatorvid or derivative thereof is administered in combination with epoprostenol. In certain embodiments, resatorvid or derivative thereof is administered in combination with recombinant human erythropoietin. In certain embodiments, resatorvid or derivative thereof is administered in combination with lactate. In certain embodiments, resatorvid or derivative thereof is administered in combination with ondansetron. In certain embodiments, resatorvid or derivative thereof is administered in combination with hyperbaric oxygen.

### EXAMPLES

### TAK-242, an Antagonist for Toll-Like Receptor 4, Protects Against Acute Cerebral Ischemia/Reperfusion Injury in Mice

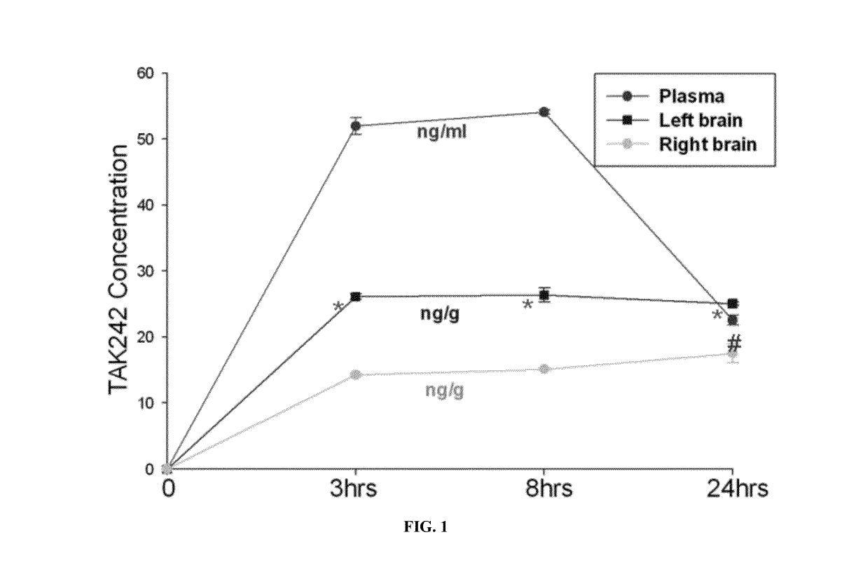

To investigate whether TAK-242 can pass through BBB and protect brain from cerebral I/R, the concentration of TAK-242 we measured in plasma and brain tissue, the ischemia-induced inflammation and subsequent brain damage was evaluated in mice treated with TAK-242. The concentration of TAK-242 in plasma increased 3 hrs after treatment, was maintained 8 hrs after treatment, and decreased at 24 hrs after treatment. Thus, TAK-242 injected i.p. can be absorbed into blood circulation and is maintained at a high level for up to 24 hrs after injection. Interestingly, the concentration of TAK-242 in brain tissue also increased after the injection, indicating that TAK-242 can pass through the BBB and be maintained in brain tissue at a high level at least 24 hrs after injection. Moreover, the concentration of TAK-242 in the ischemic hemisphere was significantly higher than that in the contralateral hemisphere (FIG. 1), indicating that cerebral ischemia increased the permeability of BBB and facilitated the diversion of TAK-242 into brain tissue.

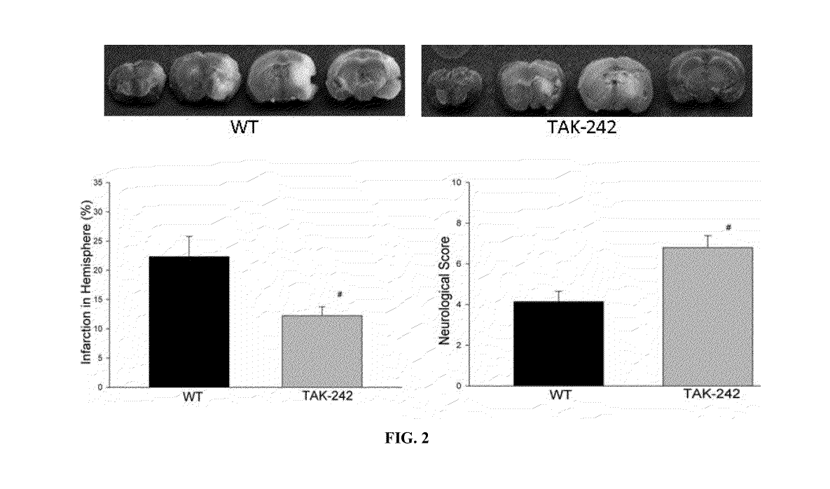

Treatment with TAK-242 1 hr after cerebral ischemia significantly reduced brain infarct size by 41% 24 hrs after cerebral I/R compared to non-treatment controls, and significantly improved neurological function as shown in FIG. 2. These results demonstrate that treatment with TAK-242 has a neuroprotective effect at the acute stage of cerebral ischemia.

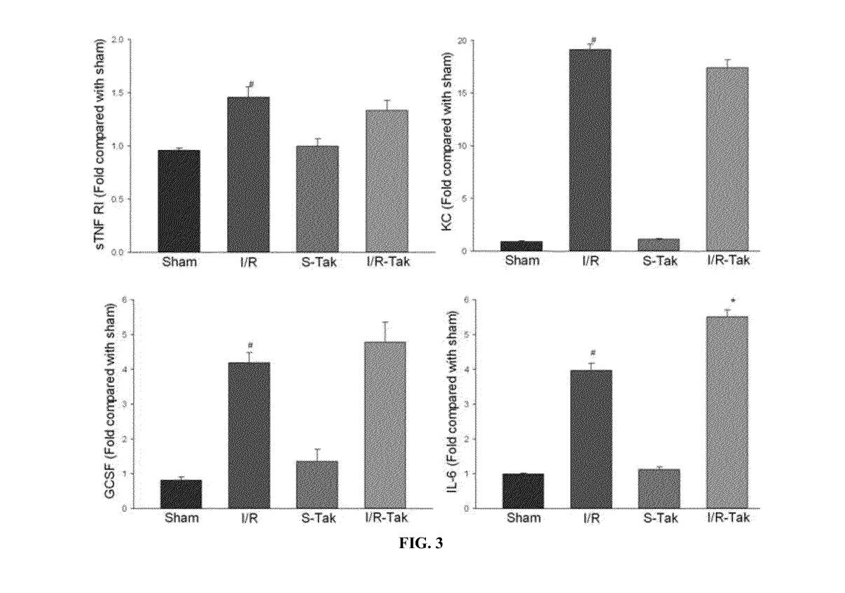

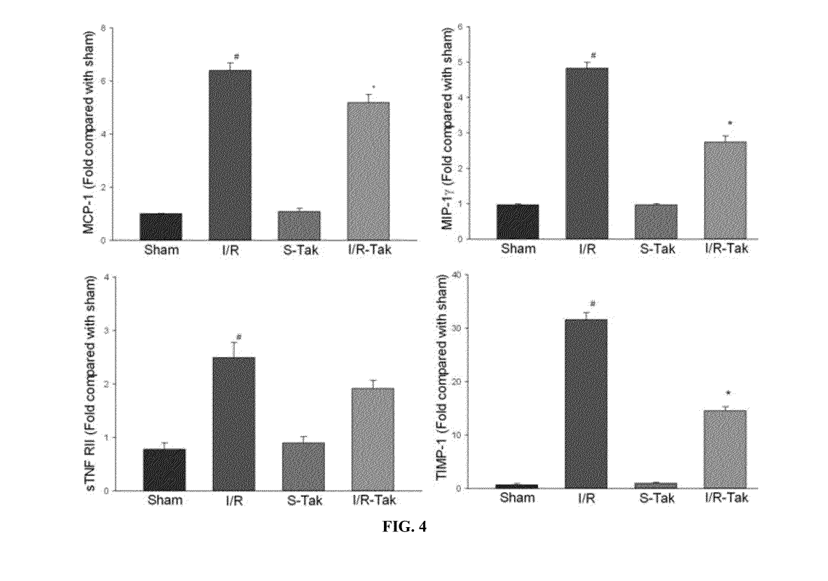

To investigate the mechanisms underlying such effect, the levels of 40 inflammatory cytokines were evaluated 6 hrs after cerebral FR using an antibody array. Our data showed that, in the 40 detected inflammatory cytokines, the levels of sTNFRI, sTNFRII, KC, GSCF, IL-6, MCP-1, MIP-1γ, and TIMP-1 significantly increased in ischemic brain compared to sham controls (p<0.05, FIGS. 3 and 4). sTNFRs are associated with infarct size and ventricular dysfunction in ST-elevation myocardial infarction. Serum levels of cytokines and C-reactive protein in acute ischemic stroke patients are related to stroke lateralization, type, and infarct volume. GCSF was found to improve memory and neurobehavior in an amyloid-β-induced experimental model of Alzheimer's disease (Prakash et al., Pharmacol Biochem Behav, 2013, 110C:46-57). However, the ability of GCSF-stimulated neutrophils to migrate into injured tissue may be impaired in traumatic brain injury. IL-6 levels were increased in the acute phase of stroke compared to healthy controls and correlated with larger stroke volume and less favorable prognosis after 1 year. IL-6 is considered a robust early marker for outcome in acute ischemic stroke. MCP-1 plays a role in inflammatory processes and contributes to the pathogenesis of myocardial infarction and ischemic stroke. Increased MIP-1γ was observed in pneumococcal meningitis, which might play a role in pneumococcal meningitis. Decreased expression of TIMP-1 is associated with the improvement of neurological function in cerebral I/R injury (Zhao et al. Brain Inj, 2013, 27(10):1190-1198). The levels of sTNFRI, sTNFRII, KC, GSCF, IL-6, MCP-1, MIP-1γ, and TIMP-1 significantly increased in ischemic brain compared with sham controls, and confirmed that these inflammatory cytokines are involved in the process of cerebral I/R injury. Importantly, ou treatment with TAK-242 significantly reduced the levels of sTNF RII, MCP-1, MIP-1γ, and TIMP-1 (p<0.05), indicating that administration of TAK-242 can inhibit the activation of these cytokines and reduce in situ inflammatory responses in the ischemic brain.

Data herein indicates that TAK-242 can pass through the BBB and it protects the brain from damage during the acute stage after cerebral I/R by mediating the expression of inflammatory cytokines. Thus, TAK-242 and derivative may be used for treating or preventing ischemic stroke.

### Materials and Methods

Animals—Fifty male mice (C57BL/6J, body weight 25˜30 g) were obtained from Jackson Laboratory and maintained in the Division of Laboratory Animal Resources at Emory University”. The experiments outlined in this manuscript conform to the Guide for the Care and Use of Laboratory Animals published by the National Institutes of Health. The animal care and experimental protocols were approved by the Emory University Committee on Animal Care. Animals were randomly assigned to four groups: sham control (S, n=4, for molecular analysis), focal cerebral ischemia/reperfusion (I/R, n=16; 5 for molecular analysis), Sham+TAK-242 (S-T, n=4, for molecular analysis), and I/R+TAK-242 (I/R-T, n=16; 5 for molecular analysis). Ten mice were used for the measurement of TAK-242 concentration in plasma and brain tissue.

Focal cerebral ischemia/reperfusion—Focal cerebral FR was induced by occlusion of the middle cerebral artery (MCAO) on the left side. Mice were subjected to anesthesia by 5.0% isoflurane and maintained by inhalation of 1.5% to 2% isoflurane driven by 100% oxygen flow. Mice were ventilated (110 breaths/min with volume 0.5 ml) and body temperature was regulated at 37.0° C. Heart rate and PO2 were monitored during surgery. Following the skin incision, the left common carotid artery (CCA), the external carotid artery (ECA), and the internal carotid artery (ICA) were carefully exposed. Microvascular aneurysm clips were applied to the left CCA and the ICA. A coated 6-0 filament (6023PK, Doccol Corp. CA, USA) was introduced into an arteriotomy hole, fed distally into the ICA and advanced 11 mm from the carotid bifurcation. The ICA clamp was removed and focal cerebral ischemia started. After ischemia for 60 min, the filament and the CCA clamp were gently removed (reperfusion starts). The collar suture at the base of the ECA stump was tightened. The skin was closed, anesthesia discontinued, and the animal allowed to recover in a pre-warmed cage. Control mice underwent a neck dissection and coagulation of the external carotid artery, but no occlusion of the MCA. Ischemia and reperfusion were confirmed by rCBF.

TAK-242 treatment—TAK-242 was dissolved in DMSO and then diluted in sterile endotoxin-free water and injected intraperitoneally (i.p., 3 mg/kg body weight) 1 hr after MCAO or sham surgical operation.

Evaluation of neurological score—Mice were scored by a blinded investigator. The scoring system included five principal tasks: spontaneous activity over a 3-min period (0-3), symmetry of movement (0-3), open-field path linearity (0-3), beam walking on a 3 cm×1 cm beam (0-3), and response to vibrissae touch (1-3). The scoring system ranged from 0 to 15, in which 15 is a perfect score and 0 is death due to cerebral I/R injury.

Measurement of TAK242 concentration in plasma and brain tissue—Three, 8 or 24 hours after the injection of TAK242, the mice were anesthetized with 5.0% isoflurane driven by 100% oxygen flow. Blood (0.95 ml) was drawn from the left ventricle and immediately mixed with 0.05 mL sodium citrate (CCS). The samples were incubated at room temperature for 30 min and centrifuged at 8000 rpm. The plasma was collected and stored at −80° C. for future use Immediately after the blood was drawn, the mice were perfused with ice-cold normal saline via the ascending aorta until the perfusion buffer was clear from the right atrium. The brains were removed and weighed. Brain tissues were homogenized with buffer, and centrifuged at 14000 rpm for 10 min. Supernatants were collected and stored at −80° C. The concentrations of TAK-242 were detected by Intertek (USA) using Liquid Chromatography with Tandem Mass Spectrometry Detection (LC-MS/MS). Briefly, TAK-242 and internal standard (Bromfenac) were extracted from 50 μL of mouse plasma or brain homogenate by liquid-liquid extraction using methyltertiary-butyl ether (MTBE). After evaporation to dryness and reconstitution, the extracts were analyzed by LC-MS/MS. Run times were approximately 5 min. The Lower Limit of Quantitation (LLOQ) for TAK-242 is 0.5 ng/ml for plasma and 2 ng/g for brain homogenate.

Assessment of cerebral infarct size—Twenty-four hours after FR, mice were sacrificed and perfused with ice-cold phosphate buffered saline (PBS) via the ascending aorta. Brains were removed and sectioned coronally into 2-mm-thick slices. The slices were stained with 2% TTC solution at 37° C. for 15 min followed by fixation with 10% formalin neutral buffer solution (pH 7.4). The infarct areas were traced and quantified with ImageJ analysis system. Unstained areas (pale color) were defined as ischemic lesions. The area of infarction and the area of both hemispheres were calculated for each brain slice. An edema index was calculated by dividing the total volume of the left hemisphere by the total volume of the right hemisphere. The actual infarct volume adjusted for edema was calculated by dividing the infarct volume by the edema index. Infarct volumes are expressed as percentage of the total brain volume±S.E.M.

Antibody array—Proteins were prepared from ischemic cerebral hemispheres. Forty inflammatory cytokines were analyzed by antibody arrays (RayBio® Cytokine Antibody Arrays—Mouse Inflammation Antibody Array G Series I). Briefly, the glass chips were air-dried for 60 min and assembled into an incubation chamber and incubation frame. Blocking buffer (100 μl) was added into each well and the glass chips were incubated at room temperature for 30 min. After decanting the blocking buffer, 100 μl of each sample was added and incubated at 4° C. overnight. The chips were washed 5 times with wash buffer I and then 2 times with wash buffer II at room temperature; 70 μl of diluted biotin-conjugated antibodies was added to each corresponding well, and the chips were incubated at 4° C. overnight. The chips were washed as previously described and 70 μl of diluted Alexa Flour 555-conjugated streptavidin was added to each subarray. The incubation chamber was covered with adhesive film and aluminum foil, and incubated at 4° C. overnight. The chips were washed 2 times with wash buffer I. The incubation frame and chamber were disassembled. The slides were taken out and placed in a 50-ml centrifuge tube, washed 2 times with wash buffer-I at room temperature for 10 min each time, washed once with wash buffer-2 for 10 min, and rinsed with distilled H2O. The water droplets were removed by centrifuge at 1,000 rpm for 3 min and then dried completely in air for at least 20 min while protected from light. The slides were scanned using a laser scanner (Axon GenePix) with a cy3 channel. The signal data was collected and analyzed by software from RayBiotech, Inc. (Atlanta, Ga., USA).

### Concentration of TAK 242 in Plasma and Brain Tissue

The concentrations of TAK-242 in plasma and brain tissue were measured at different time points after intraperitoneal injection using LC-MS/MS. The concentration of TAK-242 in plasma increased to 52.0 ng/ml 3 hrs after treatment, was maintained at 54.1 ng/ml 8 hrs after treatment, and decreased to 22.6 ng/ml 24 hrs after treatment. The concentration of TAK-242 in brain tissue increased to 26.1 ng/ml (ipsilateral) and 14.2 ng/ml (contralateral) 3 hrs after treatment, was maintained at 26.4 ng/ml (ipsilateral) and 15.1 ng/ml (contralateral) 8 hrs after treatment, and was still maintained at 25.0 ng/ml (ipsilateral) and 17.5 ng/ml (contralateral) 24 hrs after treatment (FIG. 1).

### Infarct Size and Neurological Score

Infarct size and neurological score were measured 24 hrs after cerebral I/ROur data showed that the infarct size was 21.3% in control group compared to 12.5% in the TAK-242 treated group. TAK-242 treatment significantly reduced brain infarct size by 41% compared to control mice (p<0.05, FIG. 2). The neurological score was 4.38 in the control group, and 6.73 in the TAK-242-treated group. TAK-242 treatment significantly improved neurological function by 34% compared to control mice (p<0.05, FIG. 2).

Levels of 40 inflammatory cytokines were detected 6 hrs after cerebral I/R using an antibody array. Our data showed that the levels of Soluble Tumor Necrosis Factor Receptor I (sTNFRI), Soluble Tumor Necrosis Factor Receptor II (sTNFRII), Chemokine (C-X-C motif) ligand 1 (CXCL1),

Granulocyte colony stimulating factor (GSCF), interleukin-6 (IL-6), monocyte chemotactic protein-1 (MCP-1), macrophage inflammatory protein-1γ (MIP-1γ), and tissue inhibitor of metalloproteinases 1 (TIMP-1) significantly increased in ischemic brain compared with sham controls (p<0.05, FIGS. 3 and 4). Treatment with TAK-242 significantly reduced the levels of sTNFRII, MCP-1, MIP-1γ, and TIMP-1 compared with untreated controls (p<0.05, FIGS. 3 and 4).

What we claim:

1. A method of treating cerebral ischemia comprising administering an effective amount of restorvid to a subject in need thereof.

2. The method of claim 1, wherein an effective amount is 3 mg/kg.

3. The method of claim 1, wherein restorvid is administered by injection.
